(19) United States
(12) Patent Application Publication
(10) Pub. No.: US 2004/0143289 A1
(43) Pub. Date: Jul. 22, 2004

(54) Combined hemostasis pressure applicator and dressing sheet

(76) Inventors: Byron Gale Zahler, Portland, OR (US); Thomas Raymond Tribou, West Linn, OR (US)

Correspondence Address: KEITH A. CUSHING, 4201 S. W. Vacuna St., Portland, OR, 97219, US

(21) Appl. No.: 10/349,306

(22) Filed: Jan. 22, 2003

(51) Int. Cl. A61B 17/00

(52) U.S. Cl. 606/201

(57) ABSTRACT

A hemostasis device includes a pressure applicator and a dressing sheet in stacked relation whereby a pressure source bears the combination against an access site.

### BACKGROUND OF THE INVENTION

Embodiments of the present invention find application in the context of fluid access, exchange or material delivery relative to vessels of a patient. Examples include, but are not limited to, dialysis, catheter lab, radiology, blood collection, material delivery into the blood system, and device placement in or relative to the blood system. Generally, vessel access procedures include vessel and body tissue penetration and procedures to stabilize the patient against excess or undesirable bleeding.

Following vessel access procedures, e.g., skin and vein or artery penetration by an access device such as needle or catheter or sheath, hemostasis procedures stabilize the access site against undesirable bleeding. An access device penetrates the skin, typically at angular orientation relative the skin surface. At some point below the skin surface, the access device typically penetrates a vessel, e.g., penetrates a vein or artery. In some cases, a needle element of the access device establishes the skin and vessel penetration, and a catheter element of the access device is placed to support blood or material transport. In some cases, only a needle is used as an access device. In other cases, a sheath is placed following use of a needle to establish a penetration path. Thus, a variety of procedures and access device configurations establish access relative to a blood system. The access procedure typically involves fluid coupling via the access device and in relation to the vessel. Once the access device is removed, the skin penetration site couples to the vessel penetration site along a migration channel formed by the access device, e.g., along the access device penetration path.

Dressing sheets have been used and applied directly to the skin penetration site. Dressing sheets can include a blood-clotting agent, e.g., an algae or other preparation, which promotes hemostasis, e.g., promotes clotting. Also, pads or disks have been used as applicators to bear pressure against a vessel penetration site.

### SUMMARY OF THE INVENTION

A hemostasis device includes a pressure applicator and a dressing sheet in stacked relation whereby a pressure source bears the combination against an access site.

The subject matter of the present invention is particularly pointed out and distinctly claimed in the concluding portion of this specification. However, both the organization and method of operation of embodiments of the present invention, together with further advantages and objects thereof, may best be understood by reference to the following description taken with the accompanying drawings wherein like reference characters refer to like elements.

### BRIEF DESCRIPTION OF THE DRAWINGS

For a better understanding of various embodiments of the present invention, and to show how the same may be carried into effect, reference will now be made, by way of example, to the accompanying drawings in which:

FIG. 1 illustrates partially a patient undergoing a vessel access procedure and placement of a hemostasis device according to an embodiment of the present invention.

FIG. 2 illustrates partially the patient of FIG. 1 following the vessel access procedure and illustrates operation of the hemostasis device of FIG. 1.

FIG. 3 illustrates a hemostasis device according an embodiment of the present invention similar to that of FIGS. 1 and 2 but including a band securing the device to a patient limb.

FIG. 4 illustrates a hemostasis device according an embodiment of the present invention similar to that of FIGS. 1 and 2 but used with a clamp securing the device against patient.

FIG. 5 is a top perspective exploded view of a hemostasis device.

FIG. 6 is a bottom perspective exploded view of the hemostasis device of FIG. 5.

FIG. 7 is a top perspective exploded view of a hemostasis device similar to that of FIGS. 5 and 6, but including an access port for delivering an activation agent to a dressing sheet.

FIG. 8 is a bottom exploded perspective view of the hemostasis device of FIG. 7.

FIG. 9 illustrates various sterile packages holding therein various embodiments of the present invention.

FIG. 10 is a top exploded perspective view of a hemostasis device according to an embodiment of the present invention.

FIG. 11 is a bottom exploded perspective view of the hemostasis device of FIG. 10.

FIG. 12 illustrates top perspective exploded view a hemostasis device similar to that of FIGS. 10 and 11, but including an access for delivering one or more agents to a dressing sheet.

FIG. 13 is a bottom perspective exploded view of the hemostasis device of FIG. 12.

FIG. 14 illustrates in cross section and partially a hemostasis device according to an embodiment of the present invention.

### DETAILED DESCRIPTION OF EMBODIMENTS

Embodiments of the present invention may be applied in the context of access to various body parts, e.g., cutaneous penetration and artery or vein penetration. As used herein, the term “vessel” includes reference to veins and to arteries. Access to a vessel by needle penetration and in some cases catheter placement, e.g., to deliver material into the vessel or to collect material from the vessel, includes penetration of the skin, penetration of the vessel, and penetration of body tissue intermediate the skin and vessel penetration sites. A migration channel of parted body tissue connects the skin penetration site and the vessel penetration site.

Following such access procedure and removal of the access device the patient must be stabilized against undesirable bleeding, e.g., hemostasis promptly established at the vessel penetration site, along the migration channel, and at the skin penetration site. Embodiments of the present invention may be applied to promote such hemostasis.

Two processes apply in support of hemostasis. First, indirect application of pressure at the vessel penetration site, e.g., pressure applied to the skin above the vessel penetration site. Second, application of a dressing sheet at the skin penetration site, e.g., a dressing sheet promoting hemostasis and including or receiving a hemostasis agent. A variety of hemostasis agents may be employed. For example, a liquid hemostasis agent may be applied to a simple cloth-form dressing sheet to promote hemostasis. Furthermore, in some dressing sheets a hemostasis agent, which upon receiving an activation agent, is liberated from or becomes active in its blood-clotting function. For example, one form of dressing sheet includes and alginate or other blood-clotting agent and becomes liberated or activated by application of liquid thereto, e.g., by application of a saline fluid thereto. Application of liquid, e.g., blood-clotting agent or blood clotting agent activation agent, can occur prior to, during, or after an access procedure.

When the migration channel lies at angular relation to the skin surface, the vessel penetration site and the skin penetration site are offset, e.g., not vertically aligned or substantially along a common path normal to the skin surface. When the vessel penetration site is offset relative to the skin penetration site, these stabilizing processes can occur at different locations on the skin. In other words, pressure can be applied at one point on the skin above the vessel penetration site and the dressing sheet can be applied at a laterally offset location, e.g., spaced along the skin and at the skin penetration site.

Embodiments of the present invention include in combination a pressure applicator, e.g., a pad or disk, and dressing sheet in stacked relationship relative to both the vessel penetration site and the skin penetration site. Under this aspect of embodiments of the present invention, this stacking relationship concurrently supports both a pressure application process and a dressing sheet application process. Other embodiments include a retention structure to hold or secure or to detachably secure the dressing sheet in place.

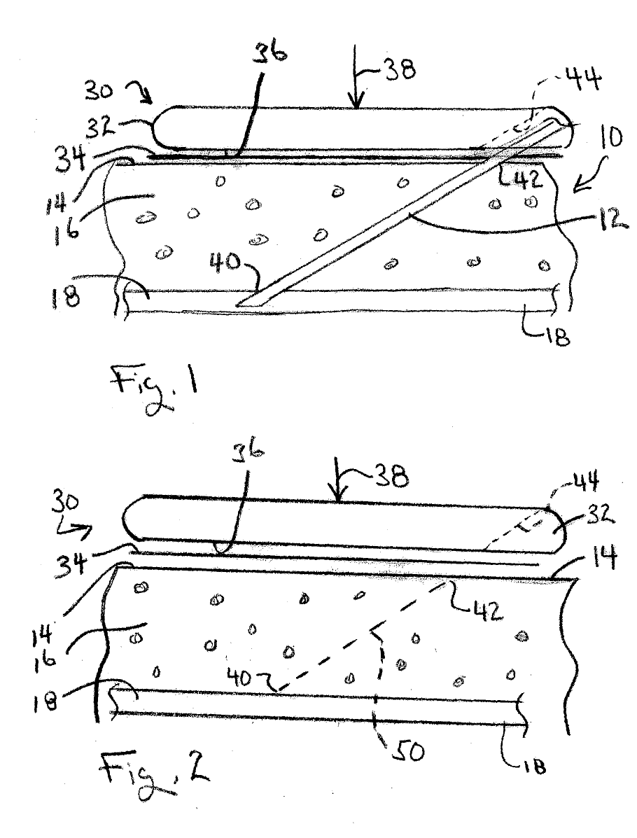

FIG. 1 illustrates partially a patient 10 undergoing a vessel access procedure wherein an access device 12, e.g., a needle or catheter or sheath, has been placed by penetrating the skin 14, intervening tissue 16, and vessel 18. Following the access procedure, access device 12 is removed as illustrated in FIG. 2. Prior to removal of access device 12, however, hemostasis device 30 can be applied to aid in establishing hemostasis. FIG. 1 illustrates placement of hemostasis device 30 just prior to removal of access device 12.

Hemostasis device 30 includes a pressure applicator 32 and a dressing sheet 34. Pressure applicator 32 may be of a rigid or semi-rigid material presenting skin-facing surface 36 for bearing pressure against skin 14. In this manner a pressure source 38 bearing against applicator 32 and toward skin 14 brings pressure directly against skin 14 and above vessel penetration site 40. Pressure source 38 can be established by a variety of methods. Pressure source 38 could be hand pressure by patient 10 or by a caregiver, but more advantageously may be established by clamp, band, weighted object, strap, or adhesive. Dressing sheet 34 can be a flexible generally thin material and may include or receive a clotting agent such as an alginate or other such agent promoting hemostasis by promoting blood clotting. Dressing sheet 34 is interposed between applicator 32 and skin 14, including at a location near skin penetration site 42. Applicator 32 can include an access device accommodation channel 44, e.g., a angular passage allowing placement of device 30 as illustrated without first removing access device 12. In this manner, hemostasis device 30 can be placed as illustrated in FIG. 1, and thereafter access device 12 can be withdrawn from patient 10.

While illustrated in FIG. 1 as lying along the surface of skin 14, sheet 34 may be urged upward into accommodation channel 44 as device 30 is brought downward against access device 12 and skin 14. In one form of device 30, however, sheet 34 can include a slit through which access device 12 passes when bringing device 30 toward and against skin 14. In other words, as device 30 is brought into position as illustrated in FIG. 1, access device 12 moves into such slit and thereby permits sheet 34 to remain in substantially planar condition, e.g., need not lie across the top of access device 12 and be thereby not urged upward into accommodation channel 44. With such a slit in sheet 34, substantially less buckling and deformation occurs and upon withdrawing access device 12, e.g., as illustrated in FIG. 2, dressing sheet 34 has better ability to lie flat against skin 14 and make better contact therewith at skin penetration site 42. Also, dressing sheet 34 need not necessarily correspond in size or area relative to the skin-facing surface 36 of disk 32. Dressing sheet 34 can be located, however, to engage skin penetration site 42 upon withdrawing access device 12. Thus, sheet 34 can be of size commensurate with that of disk 32, or can be larger or smaller than disk 32 depending on a particular implementation of device 30.

FIG. 2 illustrates patient 10 following withdrawal of access device 12. A migration channel 50 lies between vessel penetration site 40 and skin penetration site 42. Hemostasis device 30 lies generally over migration channel 50. Pressure source 38 applies pressure to bear applicator 32 against skin 14 and thereby indirectly bear pressure against vessel penetration site 40. This promotes a seal and therefore hemostasis at vessel penetration site 40. Such pressure also applies along migration channel 50, including skin penetration site 42, and promotes closure, sealing, and hemostasis therealong and thereat. Dressing sheet 34 lies in contact with skin penetration site 42 following withdrawal of access device 12. Use of a hemostasis agent, e.g., alginate or other blood-clotting agent, previously incorporated into dressing sheet 34 or added thereto during the procedure more quickly achieves hemostasis for patient 10.

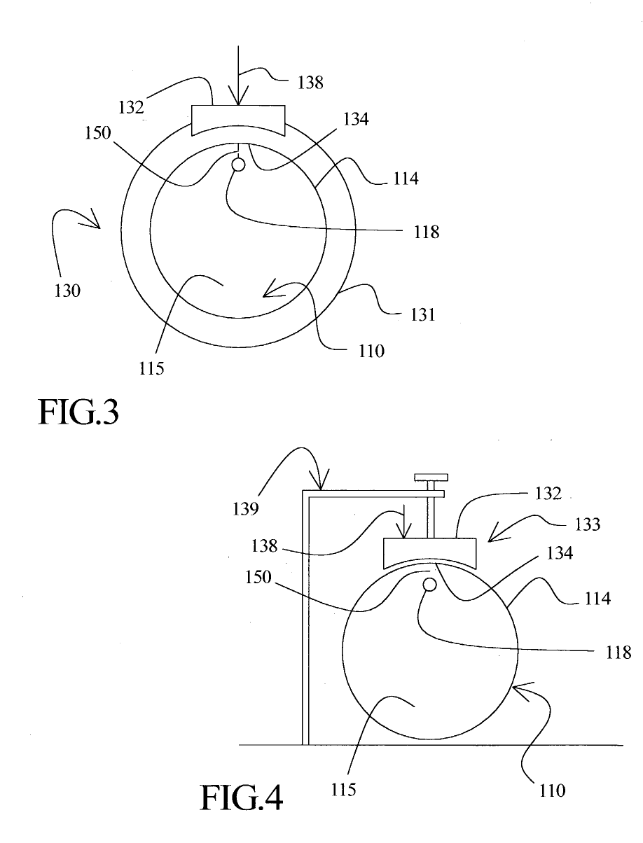

FIG. 3 illustrates a hemostasis device 130, an embodiment of the present invention similar to that of FIGS. 1 and 2 but including a band 131 securing applicator 132 and dressing sheet 134 in a secure stacked relation against skin 114 of patient 110 and in position relative to patient 110 by encircling a limb 115 of patient 110. Band 131 thereby serves as pressure source 138. Band 131 applies pressure to applicator 132 and indirectly to vessel 118 and migration channel 150 within limb 115 and to the skin penetration site at skin 114. Dressing sheet 134 can further promote hemostasis by including a suitable hemostasis agent.

FIG. 4 illustrates hemostasis device 133, an embodiment of the present invention similar to that of FIGS. 1, 2, and 3 but used with a clamp 139 securing applicator 132 and dressing sheet 134 in stacked relation against skin 114 of patient 110 and in selected position relative to patient 110. Thus, clamp 139 serves as a pressure source 138. Clamp 139 applies pressure to applicator 132 and indirectly to vessel 118 within limb 115. In addition, a migration channel 150 within limb 115 and a skin penetration site receive clamping pressure via applicator 132. Dressing sheet 134 can further promote hemostasis by including a suitable hemostasis agent.

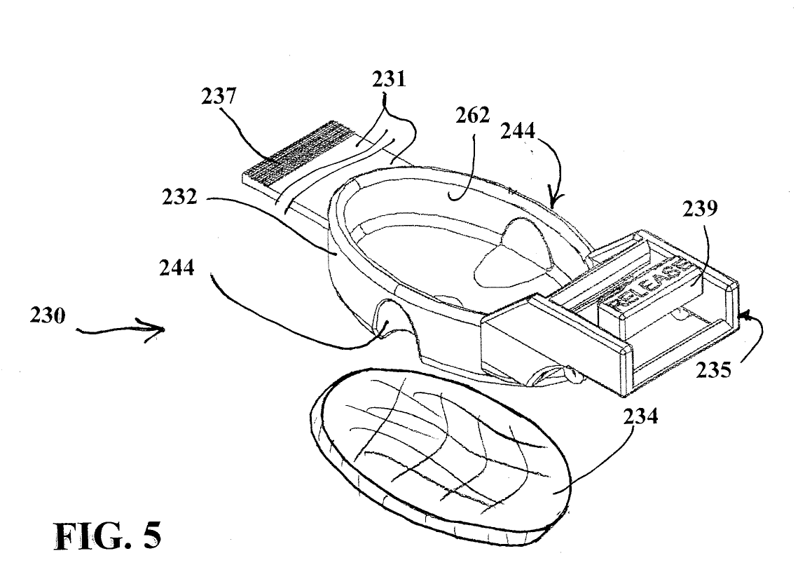

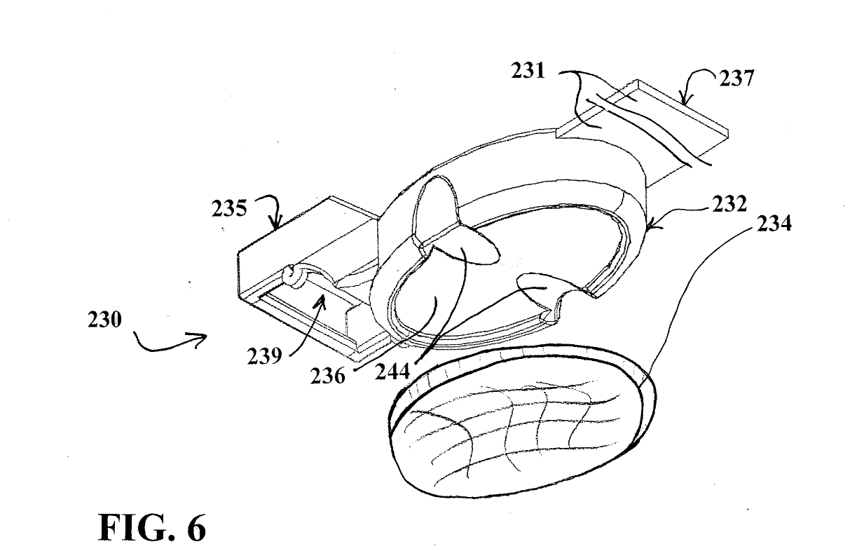

FIG. 5 is a top perspective exploded view of a hemostasis device 230. FIG. 6 is a bottom perspective exploded view of hemostasis device 230.

Hemostasis device 230 includes a pressure applicator 232. Applicator 232 includes a pair of accommodation channels 244, each accommodation channel opens, laterally and angularly upward, skin-facing surface 236. Accommodation channels 244 permit placement of device 230 over an access site prior to removal of an access device, e.g., prior to removing a needle or catheter.

A band 231 couples at one end to applicator 232 and at its distal end carries a set of serrations 237. A latch 235 couples also to applicator 232 and receives serrations 237. Latch 235 and serrations 237 cooperate to releasably capture serrations 237 and thereby provide an adjustable limb band. Surrounding a patient limb with band 231 and inserting serrations 237 into latch 235 secures device 230 at a selected location and at selected degree of pressure against a patient's skin, e.g., at a fluid access site including a skin penetration site and a vessel penetration site (not shown). Latch 235 includes a release 239 for decoupling band 231 and latch 235, e.g., when removing device 230 from a patient limb.

Skin-facing surface 236 bears toward the patient's skin when in use and includes a dressing sheet recess 250. Recess 250 locates and secures in position a dressing sheet 234. In the embodiment illustrated, sheet 234 dimensions follow that of recess 250. Dressing sheet 234 thereby sits securely within recess 250. Dressing sheet 234 can be positioned in recess 250 just prior to engaging a patient. Advantageously, however, sheet 234 can be preloaded, e.g., mounted at time of manufacture and placement in sterile packaging, in its recess 250. A variety of adhesive, clip, barb and other attachment methods and structures (not shown) can be used to retain or couple dressing sheet 234 in recess 250 following manufacture and during sterile storage and during use. In this manner device 230 can be taken from packaging and used efficiently and directly without need to place, e.g., individually manipulate, sheet 234 into stacked relation to applicator 232. Advantage also lies in making sheet 234 detachably mounted to applicator 232 as discussed hereafter.

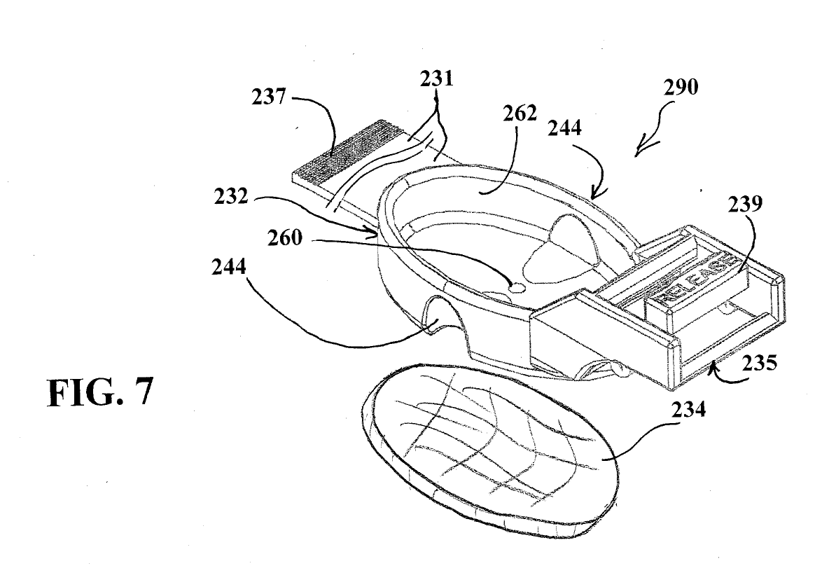

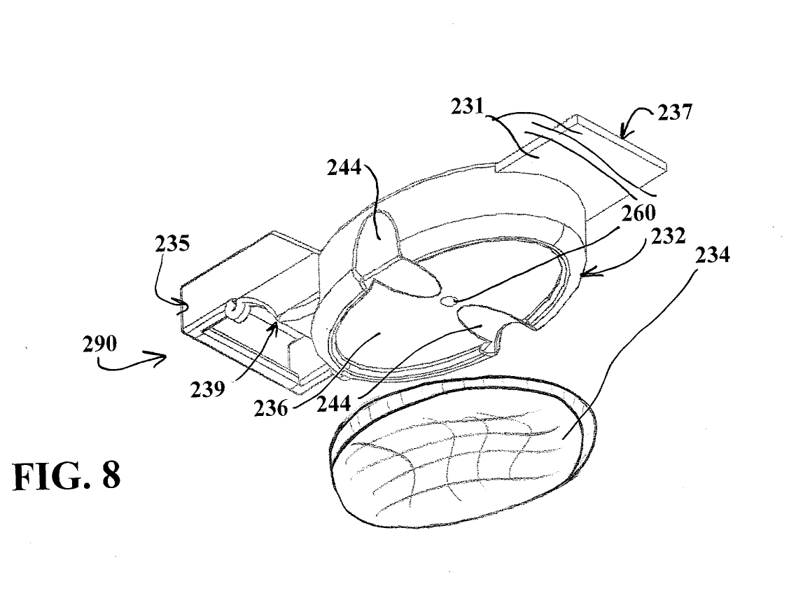

FIGS. 7 and 8 illustrate in top and bottom exploded perspective views, respectively, a hemostasis device 290 similar to device 230 and other embodiments of the present invention, but including a fluid delivery feature. Fluid, including a hemostasis agent and/or a hemostasis agent activation agent, can be applied by way of such fluid delivery feature.

More particularly, applicator 232 includes a port 260 opening into skin-facing surface 236. Port 260 provides a fluid conduit from a well 262 located opposite skin-facing surface 236. In use, with applicator 232 and dressing sheet 234 bearing against an access site (not shown), fluid placed in well 262 passes through port 260 and reaches dressing sheet 234. Sheet 234 can be activated, e.g., as by saline fluid applied to an alginate form of dressing sheet 234. A variety of blood-clotting agents can be integrated into or be applied upon a dressing sheet 234 and can be activated by delivering a suitable activation agent by way of port 260. In the alternative, dressing sheet 234 can be a simple cloth-form material which receives a fluid clotting agent by way of port 260. Dressing sheet 234 can become “slippery” when wet. Applying fluid, e.g., a blood-clotting agent or a blood-clotting agent activation agent, after positioning sheet 234 against the patient's skin aids in establishing a stable mounting of device 230 relative to the patient's skin

In other words, a dressing sheet, e.g., sheet 234, can be applied dry and can be thereafter moistened by way of a fluid port in a hemostasis device as provided under various embodiments of the present.

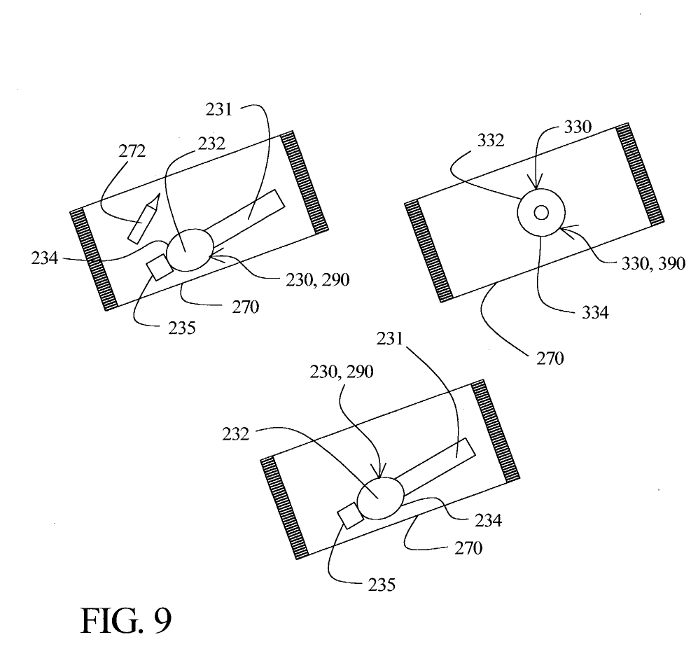

FIG. 9 illustrates various sterile packages 270 holding therein various embodiments of the present invention, e.g., a device 230 or 290, ready for use, e.g., with a dressing sheet 234 mounted in recess 250. Further, package 270 can optionally include a supply of activation agent fluid by dispenser 272. For example, dressing sheet 234 can contain alginate blood-clotting feature and dispenser 272 can include saline fluid as an activating agent relative to sheet 234.

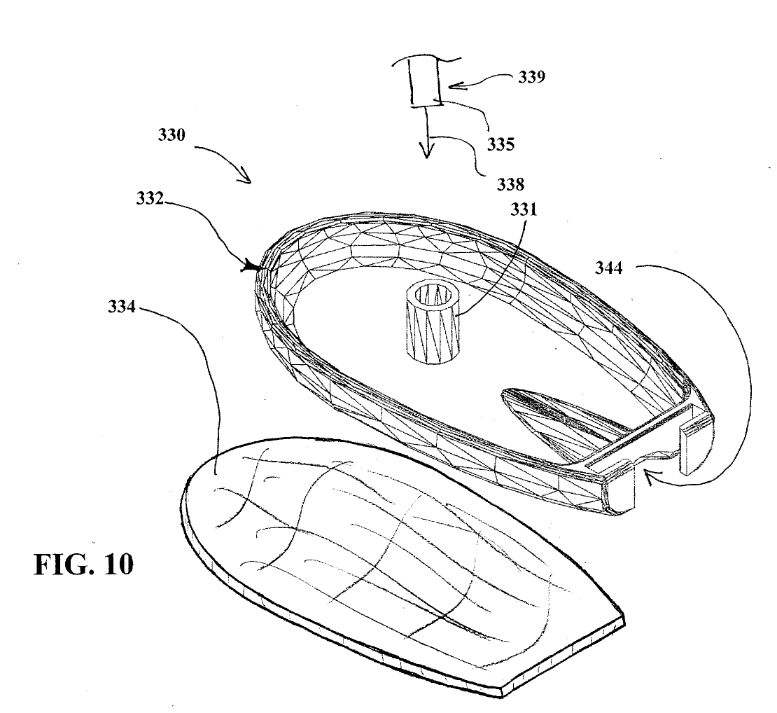

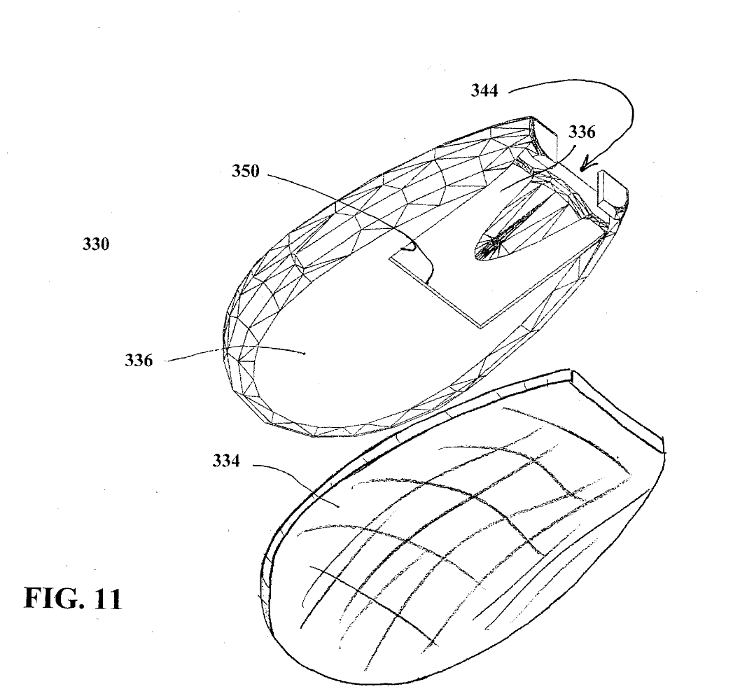

FIG. 10 is a top perspective exploded view of a hemostasis device 330. FIG. 11 is a bottom perspective exploded view of hemostasis device 330.

Hemostasis device 330 includes a pressure applicator 332. Applicator 332 includes an accommodation channel 344 opens laterally and angularly upward a skin-facing surface 336. A clamp post anchor 331 lies generally centrally within a well 362. A separate clamp mechanism 339 couples at anchor 331. For example, a clamp post 335 rests within anchor 331 and clamp mechanism 339 (shown partially) serves as a pressure source 338. A patent suitably located relative to clamp mechanism 339 can be positioned to receive by clamping force applicator 332 and sheet 334 against an access site as described hereinabove. In this manner, a selected patient location, e.g., access site, receives a selected degree of pressure. In other words, device 330 may be applied by clamping force against a patient's skin, e.g., at an access site including a skin penetration site and a vessel penetration site.

Skin-facing surface 336 bears toward the patient's skin when in use and includes a dressing sheet recess 350. Recess 350 locates and secures in position a dressing sheet 334. In the embodiment illustrated, sheet 334 follows the dimensions of recess 350 and thereby sits securely therein. Dressing sheet 334 can be positioned in recess 350 just prior to engaging a patient, or can be preloaded, e.g., mounted at time of manufacture and placement in sterile packaging, in its recess 350. A variety of adhesive, clip, barb and other coupling methods and structures can be used to retain or attach sheet 334 within recess 350 following manufacture and during sterile storage and use. In this manner device 330 can be taken from directly from packaging and applied more promptly to establish hemostasis. Advantage also lies in making sheet 334 detachably mounted to applicator 332 as discussed hereafter.

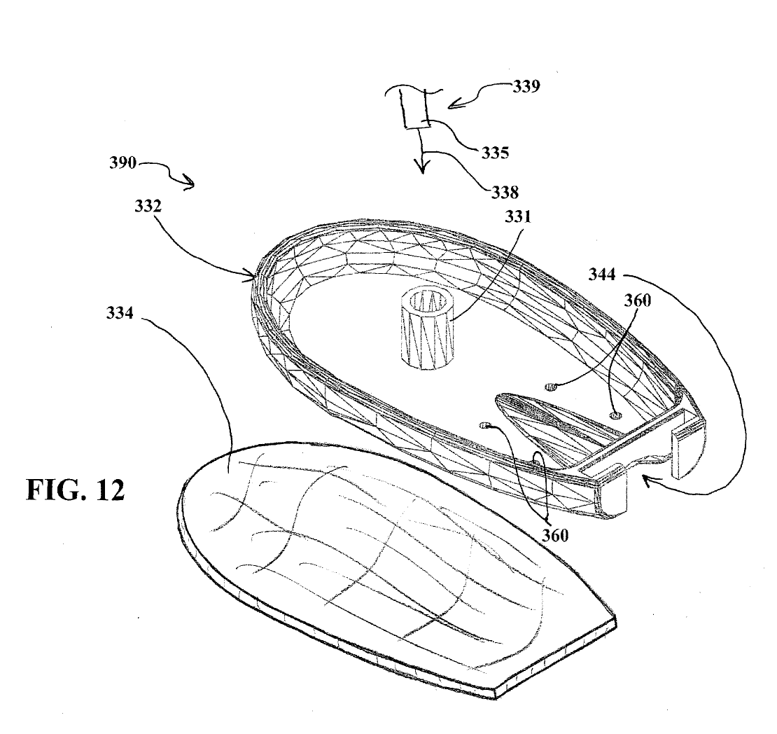

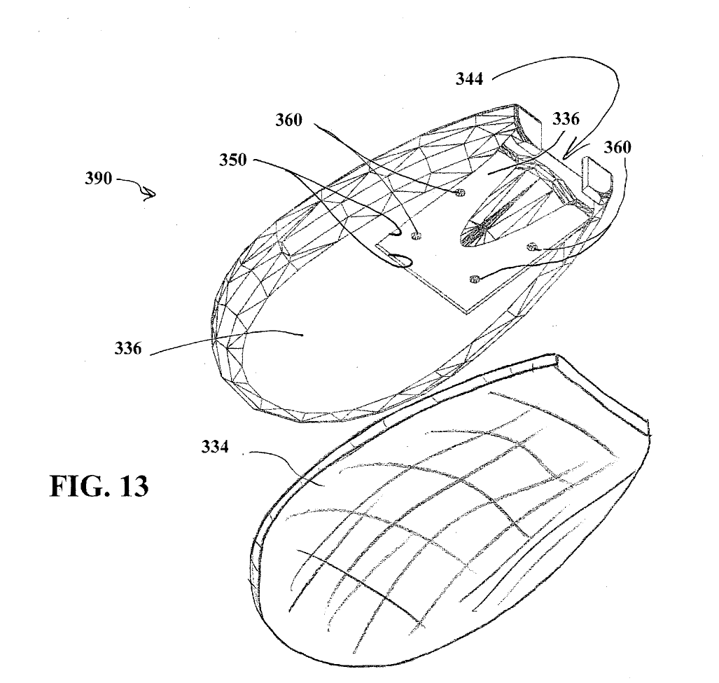

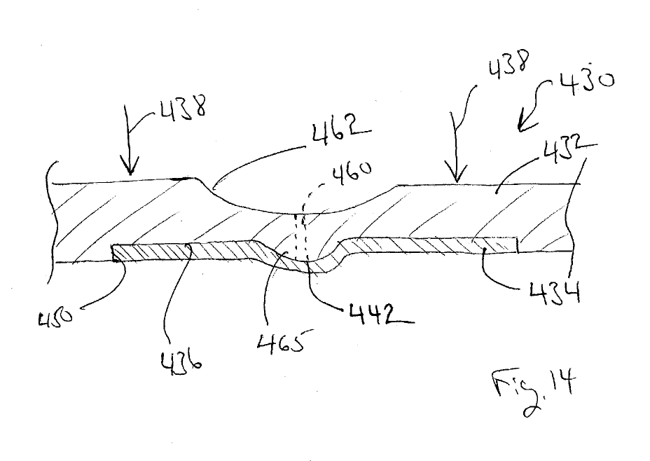

FIGS. 12 and 13 illustrate in top and bottom exploded perspective views, respectively, a hemostasis device 390 similar to device 330 and other embodiments of the present invention, but including an activating agent delivery feature. Fluid, including a hemostasis agent and/or a hemostasis agent activation agent, can be applied by way of such fluid delivery feature.

More particularly, applicator 332 includes ports 360 opening into skin-facing surface 336. Ports 360 provide a fluid conduit from a well 362 located opposite skin-facing surface 336. In use, with applicator 332 and sheet 334 bearing against an access site (not shown), activating fluid placed in well 362 travels along ports 360 and reaches dressing sheet 334. Sheet 334 can be activated, e.g., as by saline fluid applied to an alginate from of dressing sheet 334. A variety of blood-clotting agents can integrated into or be applied upon a dressing sheet 334 and can be activated by delivering a suitable activation agent by way of port 360. As may be appreciated, dressing sheet 334 can be a simple cloth-form dressing sheet and a liquid clotting agent introduced by way of ports 360. Dressing sheet 334 can become “slippery” when wet. Applying fluid, e.g., activating agent, after positioning and applying pressure against the patient's skin (not shown) aids in establishing a stable mounting of device 330 relative to the patient's skin. Sheet 334 can be applied dry and then activated by way of fluid port in a hemostasis device as provided under various embodiments of the present.

Returning to FIG. 9, a sterile package 270 can hold therein a hemostasis device, e.g., a device 330 or 390, ready for use, e.g., with a dressing sheet 334 mounted in recess 350. A supply of activation agent fluid or clotting fluid within a dispenser may be included in package 270 along with device 330.

FIG. 14 illustrates partially and in cross section a hemostasis device 430 including a pressure applicator 432 and a dressing sheet 434. As in earlier embodiments, a pressure source, e.g., pressure source 438 in FIG. 14, applies pressure against applicator 432 which in turn bears against a patient's skin (not shown in FIG. 14) in promoting hemostasis. A dressing sheet 434 resides within a recess 450 of applicator 432. Device 430 includes several features which may be incorporated into other embodiments of the present invention. For example, device 430 includes a fluid well 462 and an access port 460. Well 462 may be formed of selected volume according to a selected volume of fluid agent applied to dressing sheet 434 by way of access port 460. In other words, well 462 need only be of sufficient volume to apply sufficient fluid agent, e.g., blood clotting agent or an activation agent to a blood clotting compound or formulation within dressing sheet 434. Device 430 also includes a nipple 465 providing protrusion relative to the skin-facing surface 436 of device 430. Nipple 465 may be selectively located relative to a patient to promote hemostasis. For example, locating nipple 465 relative to, e.g., over, a skin penetration site 442 applies slightly greater pressure thereat and more quickly stabilizes skin penetration site 442. As may be appreciated, locating access port 460 along nipple 465 and allowing a flow of blood clotting agent or a flow of activation agent along access port 460 delivers such material at a skin penetration site when nipple 465 is located thereat. A variety of pressure sources 438 may be employed such as earlier-described devices including, but not limited to, bands, straps, adhesive, clamps, weighted applicators, and hand pressure.

Under embodiments of the present invention, a dressing sheet may be coupled to the pressure applicator in prepackaged fashion. In some cases, it can be helpful to provide a dressing sheet detachably mounted to the pressure applicator, e.g., to permit selective use in stacked and coupled relation relative to a pressure applicator or separately. Separating the dressing sheet allows hand placement and hand pressure against a skin penetration site as when a given applicator dimension falls short of a distance separating a given vessel penetration site and skin penetration site.

A variety of dressing sheets may be used under various embodiments of the present invention. A simple cloth-form dressing sheet may be employed wherein a liquid-form blood clotting agent may be applied, e.g., by way of access port, to encourage blood clotting. Other forms of dressing sheets may include a blood clotting agent impregnated therein, and activated or liberated by way of liquid activation agent applied thereto. Generally, dressing sheets as usable under various embodiments of the present invention can be simple cloth-form material receiving a chemical formulation or natural product used to promote clotting. Dressing sheets can come with a blood-clotting agent in some cases, and be activated by application of an additional agent thereto during use of various embodiments of the present invention, e.g., by application of fluid through access ports provided in pressure applicators according to various embodiments of the present invention.

Some dressing sheets benefit from moisture, by application of saline fluid, to liberate and activate a chemical agent or alginate agent and to promote migration thereof along the access device insertion path. Thus, a moistening step liberates or activates the agent, e.g., the algae or other such chemical, from the dressing sheet and allows migration of blood-clotting agents to the vessel penetration site. Because these dressing sheets become slippery when wet, certain difficulty has arisen in successfully stacking together a sheet and a disk and applying pressure. In other words, often the sheet due to its slippery nature when moistened will move relative to the disk and frustrate the hemostasis procedure. Under various embodiments of the present invention, however, hemostasis devices can first stabilize the dressing sheet in dry condition and thereafter apply an activating agent.

Under embodiments of the present invention, the pressure applicator includes a recess accommodating in shape the dressing sheet and thereby retaining the dressing sheet against relative movement. Furthermore, the pressure applicator may be provided with access ports from an exterior side into the sheet retention area to deliver an activation agent, e.g., saline, to the sheet after placement of the sheet and pressure applicator in stacked relation against the patient's skin. The dressing sheet and pressure applicator may brought against a patient's skin followed by application of pressure, e.g., mechanical application of pressure by strap mechanism, by clamp apparatus, or by weighted or handheld device, to stabilize the arrangement in dry condition. Thereafter, application of moisture or saline to the dressing sheet by way of the access port activates the dressing sheet.

Size and shape of various pressure applicators according to various embodiments of the present invention can include sufficient size to cover a distance separating laterally the vessel penetration site and the skin penetration site, and generally sufficient to cover the access site as a whole. The lateral separation between a vessel penetration site and a skin penetration site varies as a function of the distance between the skin and the vessel as well as the particular angle of insertion for the needle or other penetrating access device. Thus, various embodiments of the present invention can include length dimensions sufficient to cover an access site so as to concurrently apply pressure over the vessel penetration site and make contact between the dressing sheet and the skin penetration site.

While particular shapes have been illustrated herein for dressing sheets, it will be understood that the shape of a particular dressing sheet need not follow those particular shapes illustrated herein. For example, devices 230 and 290 include an oval-shape for dressing sheet 234, but such dressing sheet could be of an arbitrary shape, e.g., including a square or rectangular shape. Generally, it is advantageous to provide a recess of corresponding shape, e.g., a recess in a pressure applicator corresponding in size and shape or following the dimensions of the dressing sheet whereby upon locating the dressing sheet within the recess the dressing sheet enjoys a degree of stability and security therein especially when used during a hemostasis procedure as described herein.

In general use, pressure applicators under embodiments of the present invention can be of sufficient size to span a distance between a skin penetration site and a vessel penetration site. However, in some instances of use such distance may exceed the dimensions of the pressure applicator and under such circumstances it is helpful to remove the dressing sheet from the pressure applicator to permit hand placement and pressure against the dressing sheet and relative to the skin penetration site, leaving the pressure applicator in place relative to the vessel penetration site.

Also, in some circumstances, blood-clotting agents or blood-clotting agent activating agents may be applied prior to, during, or after an access procedure. In other words, the timing of applying a blood clotting agent or blood-clotting activation agent, e.g., in liquid form by way of access ports in a pressure applicator as provided under various embodiments of the present invention, can vary relative to the timing of an access procedure generally.

It will be appreciated that the present invention is not restricted to the particular embodiments that have been described and illustrated, and that variations may be made therein without departing from the scope of the invention as found in the appended claims and equivalents thereof.

What is claimed is:

1. A method of establishing hemostasis at an access site, the method comprising:
stacking a pressure applicator and a dressing sheet;
placing said dressing sheet against said access site; and
applying pressure against said pressure applicator toward said access site.

2. A method according to claim 1 wherein said method comprises removing said pressure applicator and said dressing sheet in coupled and stacked relation from a sterile package.

3. A method according to claim 2 wherein said method includes said pressure applicator and said dressing sheet being coupled.

4. A method according to claim 3 wherein said pressure applicator includes a dressing sheet recess receiving said dressing sheet therein.

5. A method according to claim 4 wherein said recess and said dressing sheet follow similar dimensions.

6. A method according to claim 4 wherein said recess and said dressing sheet have sufficiently similar dimensions to stabilize said dressing sheet relative to said pressure applicator when said dressing sheet is located within said recess.

7. A method according to claim 1 wherein said applying pressure step comprises surrounding a patient limb by band coupled to said pressure applicator.

8. A method according to claim 1 wherein said applying pressure step comprises applying clamping pressure to said pressure applicator and toward said access site.

9. A method according to claim 1 further comprising passing fluid through said pressure applicator and into said dressing sheet.

10. A method according to claim 1 wherein said access site includes a vessel penetration site and a skin penetration site, said pressure applicator being of sufficient size to span said vessel penetration site and said skin penetration site.

11. A method according to claim 1 wherein said dressing sheet is removably dismountable relative to said pressure applicator.

12. A hemostasis device comprising:
a pressure applicator;
a dressing sheet; and
a pressure source.

13. A device according to claim 12 wherein said applicator includes a recess and said dressing sheet lies within said recess.

14. A device according to claim 13 wherein said recess corresponds in dimensions to dimensions of said dressing sheet.

15. A device according to claim 13 wherein said dressing sheet is removably dismountable relative to said pressure applicator.

16. A device according to claim 12 wherein said dressing sheet couples to said pressure applicator.

17. A device according to claim 16 wherein said applicator includes a recess and said dressing sheet lies coupled within said recess.

18. A device according to claim 16 wherein said dressing sheet is detachably mounted relative to said pressure applicator.

19. A device according to claim 12 wherein said pressure source comprises at least one of an adjustable limb band and a clamping mechanism.

20. A device according to claim 12 wherein said pressure applicator includes a fluid port.

21. A device according to claim 20 wherein said port couples an outward-facing surface of said pressure applicator and a skin-facing surface of said pressure applicator.

22. A device according to claim 21 wherein said dressing sheet bears against said skin-facing surface.

23. A device according to claim 22 wherein said skin-facing surface forms in part a recess holding said dressing sheet.

24. A device according to claim 23 wherein said recess corresponds in dimensions to dimensions of said dressing sheet.

25. A hemostasis device comprising:
a pressure applicator, said applicator including a recess;
a dressing sheet, said dressing sheet residing within said recess and coupled to said pressure applicator; and
a package holding said pressure applicator and said dressing sheet.

26. A device according to claim 25 wherein said dressing sheet includes a blood-clotting agent.

27. A device according to claim 25 where said device include an adjustable band coupled to said pressure applicator.

28. A device according to claim 25 wherein said device includes an anchor for receiving a clamping force when said device is placed in service outside said package.

29. A device according to claim 25 wherein said pressure applicator includes a, fluid port including an opening communicating with said dressing sheet.

30. A device according to claim 25 wherein said packaging includes at least one of a supply of blood-clotting agent and a blood-clotting agent activation agent.

31. A device according to claim 30 wherein said blood-clotting activating agent is cooperative with said blood-clotting agent to at least one of activate and liberate said blood-clotting agent when applied thereto.

32. A device according to claim 25 wherein said package maintains said pressure applicator and said dressing sheet in sterile condition while held therein.

33. A device according to claim 25 wherein said recess and said dressing sheet correspond in respective dimensions.
